(19) United States
(12) Patent Application Publication
(10) Pub. No.: US 2011/0208083 A1
(43) Pub. Date: Aug. 25, 2011

(54) DEVICE AND METHOD FOR ADJUSTING IMPEDANCE BASED ON POSTURE OF A PATIENT

(75) Inventor: Dan E. Gutfinger, Agoura Hills, CA (US)

(73) Assignee: PACESETTER, INC., Sylmar, CA (US)

(21) Appl. No.: 12/712,003

(22) Filed: Feb. 24, 2010

(51) Int. Cl. A61B 5/055

(52) U.S. Cl. 600/547

(57) ABSTRACT

An implantable medical device includes electrodes that are configured to be positioned within at least one of a heart and a chest wall of a patient. The device also includes an impedance measurement module, a patient position sensor, and a correction module. The impedance measurement module measures an impedance vector between a predetermined combination of the electrodes. The patient position sensor determines at least one of a posture and an activity level of the patient. The correction module adjusts the impedance vector based on the at least one of the posture and the activity level of the patient.

### FIELD OF THE INVENTION

Embodiments described herein generally pertain to implantable medical devices and more particularly to methods and devices that obtain impedance vectors between electrodes positioned within a heart and/or chest wall.

### BACKGROUND OF THE INVENTION

An implantable medical device (IMD) is implanted in a patient to monitor, among other things, electrical activity of a heart and to deliver appropriate electrical therapy, as required. IMDs include pacemakers, cardioverters, defibrillators, implantable cardioverter defibrillators (ICD), and the like. The electrical therapy produced by an IMD may include pacing pulses, cardioverting pulses, and/or defibrillator pulses to reverse arrhythmias (for example, tachycardias and bradycardias) or to stimulate the contraction of cardiac tissue (for example, cardiac pacing) to return the heart to its normal sinus rhythm. These pulses are referred to as stimulus or stimulation pulses.

IMDs may monitor electrical characteristics of the heart to identify or classify cardiac behavior and to estimate physiological parameters of the heart. For example, some known IMDs measure intracardiac and intrathoracic impedance vectors between combinations of electrodes in the heart and/or chest wall to estimate left atrial pressure (LAP) in the heart. As the left atrium of the heart fills with fluid and the LAP increases, the impedance measured between two electrodes and along a vector that traverses the left atrium may decrease. Conversely, as the fluid level in the left atrium drops, the LAP may decrease and the impedance vector through the left atrium may increase.

In order to use intracardiac and intrathoracic impedance vectors to estimate LAP, the IMD may need to be calibrated so that a measured impedance vector may be accurately transformed into a corresponding estimate of LAP. Additionally, the IMD may be unable to compensate for changes in the posture of the patient because such changes can produce changes in the interelectrode spacing and geometry that may impact the measured impedance. For example, when a patient changes posture from a supine to an upright standing position an acute change in the interelectrode spacing may occur in combination with the expected decrease in the intracardiac and intrathoracic fluid volume associated with this posture maneuver. The acute change in interelectrode spacing may cause the measured impedance to either increase or decrease or not change at all. The acute decrease in intracardiac and intrathoracic fluid volume will cause the measured impedance to increase since impedance is inversely proportional to fluid volume. The overall effect of the acute change in interelectrode spacing and intracardiac/intrathoracic fluid volumes may cause the impedance measurement to either acutely increase or decrease depending on the relative magnitude and direction of the change associated with the change in interelectrode spacing. In either situation, the impedance vectors may provide an unreliable indicator of the LAP if the algorithm utilized to transform the measured impedance into an estimate of LAP did not compensate for changes in impedance that are a consequence of posture dependent rather than fluid volume dependent changes in interelectrode spacing and geometry.

A need exists for a device and method for adjusting impedance vectors or measurements to account for changes in interelectrode spacing and geometry that occur after a patient changes positions or postures.

### SUMMARY

In one embodiment, an implantable medical device is provided. The implantable medical device includes electrodes that are configured to be positioned within at least one of a heart and a chest wall of a patient. The device also includes an impedance measurement module, a patient position sensor, and a correction module. The impedance measurement module measures an impedance value (or vector) between a predetermined combination of the electrodes. The patient position sensor determines at least one of a posture and an activity level of the patient. The correction module adjusts the impedance value (or vector) based on the at least one of the posture and the activity level of the patient.

In another embodiment, a method for adjusting an impedance value (or vector) obtained by a medical device is provided. The method includes measuring the impedance value using a predetermined combination of electrodes that are positioned in at least one of a heart and a chest wall of a patient and determining at least one of a posture and an activity level of the patient when the impedance value is measured. The method also includes adjusting the impedance value based on the at least one of the posture and the activity level of the patient.

### BRIEF DESCRIPTION OF THE DRAWINGS

The drawings illustrate generally, by way of example, but not by way of limitation, various embodiments discussed in the present document.

FIG. 1 illustrates an IMD that is coupled to a heart of a patient in accordance with one embodiment.

FIG. 2 is a schematic diagram of the IMD and the heart shown in FIG. 1 when the patient is in a supine position in accordance with one embodiment.

FIG. 3 is a schematic diagram of the IMD and the heart shown in FIG. 1 when the patient is in an upright position.

FIG. 4 is a flowchart of a method for adjusting impedance vectors based on changing postures of a patient in accordance with one embodiment.

FIG. 5 illustrates a block diagram of exemplary internal components of the IMD shown in FIG. 1 in accordance with one embodiment.

FIG. 6 illustrates a functional block diagram of an external programming device shown in FIG. 5 in accordance with one embodiment.

FIG. 7 illustrates a distributed processing system in accordance with one embodiment.

FIG. 8 illustrates a block diagram of exemplary manners in which embodiments of the present invention may be stored, distributed and installed on a tangible and non-transitory computer-readable medium.

### DETAILED DESCRIPTION OF THE INVENTION

In the following detailed description, reference is made to the accompanying drawings which form a part hereof, and in which are shown by way of illustration specific embodiments in which the present invention may be practiced. These embodiments, which are also referred to herein as “examples,” are described in sufficient detail to enable those skilled in the art to practice the invention. It is to be understood that the embodiments may be combined or that other embodiments may be utilized, and that structural, logical, and electrical variations may be made without departing from the scope of the present invention. The following detailed description is, therefore, not to be taken in a limiting sense, and the scope of the present invention is defined by the appended claims and their equivalents. In this document, the terms “a” or “an” are used, as is common in patent documents, to include one or more than one. In this document, the term “or” is used to refer to a nonexclusive or, unless otherwise indicated. In this document the term “impedance vector” refers to intracardiac and/or intrathoracic impedance measurements derived from two or more electrodes positioned within the heart and/or chest wall. In this document the term “admittance” is used to denote the reciprocal of impedance.

In accordance with certain embodiments, methods and devices are provided for adjusting impedance vectors obtained between predetermined combinations of electrodes positioned within a heart and/or chest wall of a patient. An impedance vector represents an impedance measurement obtained along a path extending between the electrodes used to obtain the impedance measurement. The impedance vectors are adjusted in order to compensate for changes in the impedance measurements that are caused or affected by posture dependent changes in the inter-electrode spacing and/or geometry between the electrodes used to obtain the impedance measurements. The changes in the inter-electrode spacing and/or geometry between the electrodes may be caused by a shift or change in the posture of the patient independent of changes in intracardiac and intrathoracic fluid volume. The adjustments to the impedance measurements may prevent the changing posture of the patient from causing inaccurate estimates of various physiological parameters of the patient, such as left atrial pressure (LAP) that is derived or based on the impedance measurements.

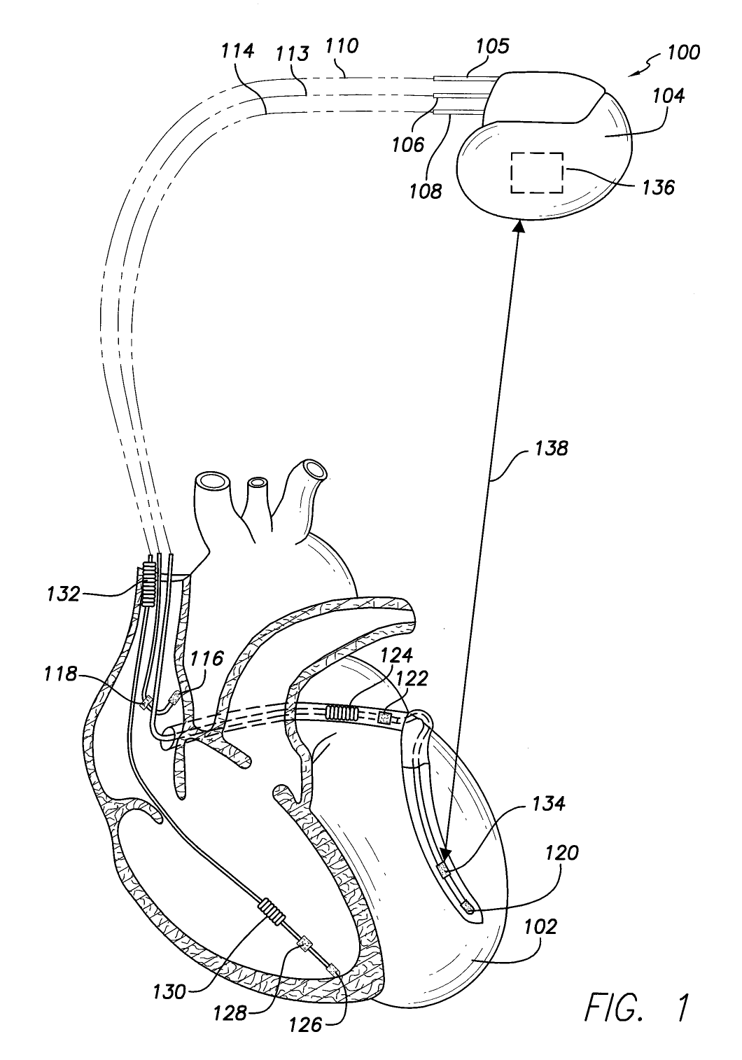

FIG. 1 illustrates an IMD 100 that is coupled to a heart 102 of a patient in accordance with one embodiment. The IMD 100 may be a cardiac pacemaker, an ICD, a defibrillator, an ICD coupled with a pacemaker, and the like, implemented in accordance with one embodiment of the present invention. The IMD 100 may be a dual-chamber stimulation device capable of treating both fast and slow arrhythmias with stimulation therapy, including cardioversion, defibrillation, and pacing stimulation, as well as capable of detecting heart failure, evaluating its severity, tracking the progression thereof, and controlling the delivery of therapy and warnings in response thereto. As explained below in more detail, the IMD 100 may be controlled to obtain impedance or admittance vectors between predetermined combinations of electrodes 104, 116, 118, 120, 122, 124, 126, 128, 130, 132, 134 positioned within the heart 102 and adjust the impedance or admittance vectors based on the posture of the patient.

The IMD 100 includes a housing 104 that is joined to receptacle connectors 105, 106, 108 that are connected to a right ventricular (RV) lead 110, a right atrial (RA) lead 112, and a coronary sinus lead 114, respectively. The IMD 100 may be located in a patient's chest wall. The leads 110, 112, 114 may be located at various locations, such as an atrium, a ventricle, or both to measure physiological parameters of the heart 102. One or more of the leads 110, 112, 114 detect IEGM signals that form an electrical activity indicator of myocardial function over multiple cardiac cycles. To sense atrial cardiac signals and to provide right atrial chamber stimulation therapy, the RA lead 112 is joined with an atrial tip electrode 116, which typically is implanted in the right atrial appendage, and an atrial ring electrode 118. The coronary sinus lead 114 receives atrial and ventricular cardiac signals and delivers left ventricular pacing therapy using at least a left ventricular tip electrode 120, delivers left atrial pacing therapy using at least a left atrial ring electrode 122, and delivers shocking therapy using at least a left atrial coil electrode 124. The coronary sinus lead 114 also includes a left ventricular ring electrode 134 that is disposed between the LV tip electrode 120 and the LV ring electrode 122. The RV lead 110 has right ventricular tip electrode 126, a right ventricular ring electrode 128, a right ventricular coil electrode 130, and an SVC coil electrode 132. The RV lead 110 is capable of receiving cardiac signals, and delivering stimulation in the form of pacing and shock therapy to the right ventricle. The RV coil electrode 130 may be used as a defibrillation electrode. For purposes of measuring impedance vectors between predetermined combinations of the electrodes 116, 118, 120, 122, 124, 126, 128, 130, 132, 134 (as described below), the housing 104 of the IMD 100 may be referred to as an electrode.

In the illustrated embodiment, the IMD 100 includes a patient position sensor 136. The patient position sensor 136 may be disposed within the housing 104 or may be communicatively coupled with the IMD 100. The patient position sensor 136 is a device that determines a position or orientation of the sensor 136. The sensor 136 may include a multi-axis accelerometer that determines the orientation of the IMD 100. As described below, the output of the sensor 136 may be used to determine the posture or position of the patient along with an activity level. For example, with respect to posture, the sensor 136 may be used to determine if the patient is in one or more of the following positions: (i) upright, or standing upright, (ii) supine, or laying on his or her back, (iii) prone, or laying on his or her stomach, (iv) right side down, or laying on his or her right side or arm, (v) left side down, or laying on his or her left side or arm, or (vi) a combination of any of the previously listed positions. A combination of positions that is detected by the sensor 136 may be used to determine if the patient is laying between a supine and right side down posture, or between a prone and a right side down posture. The sensor 136 may be used to determine an activity level of the patient by determining if the patient has recently switched or changed postures or position and/or continues to switch or change postures or positions.

The IMD 100 may measure one or more physiologic parameters of the heart 102 in order to monitor a condition of the heart 102. For example, the IMD 100 may obtain impedance or admittance vectors between predetermined combinations of the electrodes 104, 116, 118, 120, 122, 124, 126, 128, 130, 132, 134 in order to monitor LA pressure (LAP) or intracardiac pressures, ischemia of the heart 102, cardiac output, LA wall velocity, cardiac heart failure indices, the beginning of pulmonary edema, hemodynamic parameters, levels of fluid accumulation, and the like.

An impedance vector is obtained by the IMD 100 between any two or more of the electrodes 104, 116, 118, 120, 122, 124, 126, 128, 130, 132, 134. The impedance vector may be represented as the impedance measured along a path (generally a linear path) between at least two points. One or more impedance measurements obtained by the IMD 100 may extend through the heart 102. The impedance vectors that extend through the heart 102 represent the impedance of the myocardium and the blood in the heart 102 along the paths of the impedance vectors. By way of example only, the IMD 100 may measure an impedance of the heart 102 along an impedance vector 138. As shown in FIG. 1, the impedance vector 138 extends between the LV ring electrode 134 and the housing 104 of the IMD 100. Alternatively, the IMD 100 may measure additional or different impedance vectors between any two or more combinations of the electrodes 116, 118, 120, 122, 124, 126, 128, 130, 132, 134 and/or the housing 104. The impedance measured along the impedance vector 138 may be expressed in terms of ohms. Alternatively, the impedance may be expressed as an admittance measurement. The admittance may be inversely related to the impedance. By way of example only, the admittance along the impedance vector 138 may be represented as:

$$A = \frac{1000}{Z} \qquad ( Eqn . 1 )$$

where “A” represents admittance in terms of 1/mΩ and “Z” represents the impedance measurement in terms of ohms (Ω).

The impedance measured along the impedance vector 138 may vary based on a variety of factors, including the amount of fluid in one or more chambers of the heart 102 and/or thoracic space. As a result, the impedance measurement may be indicative of LAP. As more blood fills the left atrium and pulmonary veins, the LAP increases. Blood can be more electrically conductive than air and/or the myocardium of the heart 102 along the impedance vector 138. Consequently, as the amount of blood in the left atrium increases, the LAP increases and the impedance measured along the impedance vector 138 may decrease. Conversely, decreasing LAP may result in the impedance measurement increasing as there is less blood in the left atrium and pulmonary veins.

But, inter-electrode spacing also may affect the impedance measurements. For example, changes in posture of a patient from a supine position, such as supine, prone, right side down, left side down, or a combination thereof, to an upright standing position may result in changes in the distance between the LV ring electrode 134 and the housing 104 of the IMD 100. Additionally, activity of a patient may vary the distance between electrodes 104, 116, 118, 120, 122, 124, 126, 128, 130, 132, 134. For example, movement of the patient may result in changes in the distance between the LV ring electrode 134 and the housing 104.

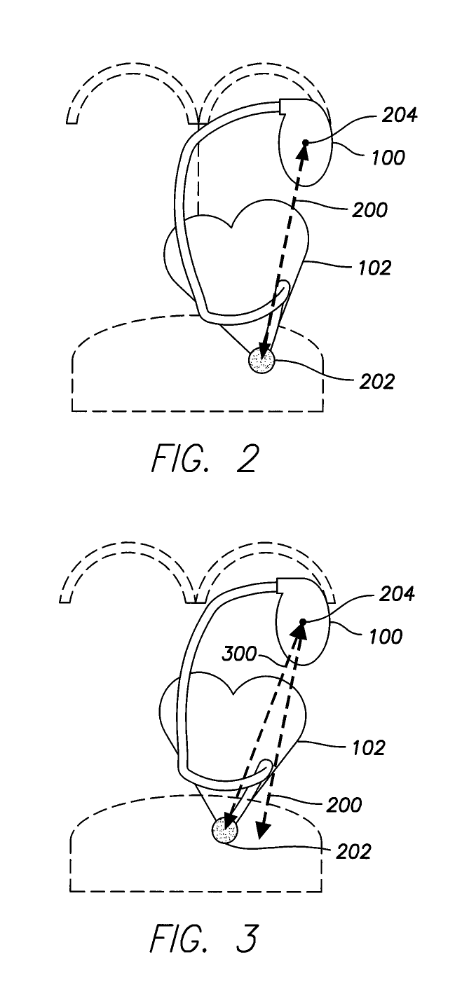

FIG. 2 is a schematic diagram of the IMD 100 and the heart 102 when the patient is in a supine position in accordance with one embodiment. As shown in FIG. 2, an impedance vector 200 extends from an electrode 202 to the IMD 100. The electrode 202 may be the LV ring electrode 134 (shown in FIG. 1) such that the impedance vector 200 may extend from the LV ring electrode 134 to a common point 204 on the housing 104 (shown in FIG. 1) of the IMD 100. Alternatively, the electrode 202 may be a different electrode 116, 118, 120, 122, 124, 126, 128, 130, 132 (shown in FIG. 1). When the patient moves from the supine position represented in FIG. 2 to another position or posture, the relative positions of the electrode 202 and the IMD 100 may change. Activity of the patient also may cause the relative positions of the electrode 202 and IMD 100 to change.

FIG. 3 is a schematic diagram of the IMD 100 and the heart 102 when the patient is in an upright standing position. As shown in FIG. 3, an impedance vector 300 extends between the electrode 202 and the common point 204 of the IMD 100. While both the impedance vectors 200, 300 extend between the electrode 202 and the common point 204 of the IMD 100, the impedance vectors 200, 300 are oriented along different directions. The impedance vectors 200, 300 are oriented along different directions due to the change in posture of the patient. The changing posture from supine posterior to upright causes the electrode 202 to move relative to the IMD 100. This may occur as a consequence of the heart 102 dropping down within the thoracic cavity when the patient stands upright, while the IMD 100 that is attached to the chest wall remaining relatively fixed. As a result, the impedance vector 200 shifts to the impedance vector 300. If the impedance vectors 200, 300 do not extend over the same distance and paths through the heart 102, the impedance measurements obtained over the impedance vectors 200, 300 may differ.

In order to compensate for the change in the spacing or geometry between the electrode 202 and the IMD 100 and the shift in the impedance vector 200 to the vector 300, the IMD 100 may apply an offset factor β to impedance measurements obtained along the impedance vector 200 or 300. The offset factor β is applied to impedance vectors 200, 300 in order to reduce or eliminate the impact of a changing posture of the patient on the impedance vectors 200, 300. As the impact of posture on the impedance vectors 200, 300 is reduced, the accuracy of physiologic parameters such as LAP derived from the impedance vectors 200, 300 may be increased. The offset factor β is derived based on impedance vectors 200, 300 measured between two electrodes 104, 116, 118, 120, 122, 124, 126, 128, 130, 132 (shown in FIG. 1) at different first and second positions, such as a supine posture and an upright standing posture. The offset factor β may then be applied to impedance vectors 200, 300 measured.

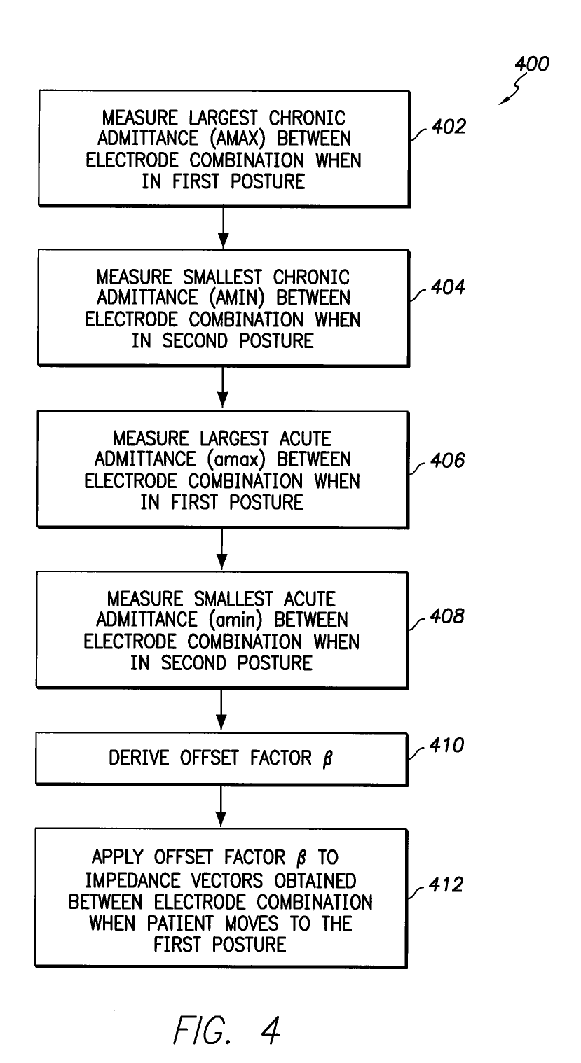

FIG. 4 is a flowchart of a method 400 for adjusting impedance vectors based on changing postures of a patient in accordance with one embodiment. The method 400 determines an offset factor β that can be applied to impedance vectors that are measured between a predetermined combination of electrodes 104, 116, 118, 120, 122, 124, 126, 128, 130, 132, 134 (shown in FIG. 1) for a change in the patient's position from a first posture to a second posture. The method 400 may be repeated several times to determine additional offset factors β for different combinations of electrodes 104, 116, 118, 120, 122, 124, 126, 128, 130, 132, 134 and/or different changes in position.

At 402, a supine chronic admittance (A_{S}) is measured between a predetermined combination of electrodes 104, 116, 118, 120, 122, 124, 126, 128, 130, 132, 134 (shown in FIG. 1) when the patient is in the position of a first posture. The supine chronic admittance A_{S} may be obtained in a chronic ambulatory setting by measuring the impedance vector between the predetermined combination of electrodes 104, 116, 118, 120, 122, 124, 126, 128, 130, 132, 134 after the patient has moved to the first posture for a sufficiently long time period that fluids within the patient's body have reached a steady state. For example, the supine chronic admittance A_{S} may be measured after a sufficient time to allow the fluid in the various chambers of the heart 102 (shown in FIG. 1) and other thoracic chambers to reach a steady state after the patient has moved to the first posture. In one embodiment, the first posture is a supine position, but may also be a prone position, a right side down position, or a left side down position.

The supine chronic admittance A_{S} may be measured by measuring the impedance vector between the predetermined combination of electrodes 104, 116, 118, 120, 122, 124, 126, 128, 130, 132, 134 after the patient have moved to the first posture, such as a supine position, and generally remained in the first posture for at least four hours. Alternatively, the supine chronic admittance A_{S} may be obtained after the patient has moved to the first posture for a different time period, such as thirty minutes, one hour, two hours, five hours, and the like.

The supine chronic admittance A_{S} may be measured as the smallest impedance vector between the predetermined combination of electrodes 104, 116, 118, 120, 122, 124, 126, 128, 130, 132, 134 (shown in FIG. 1) that is measured over a time window. The IMD 100 (shown in FIG. 1) may periodically measure the impedance vector between the predetermined combination of electrodes 104, 116, 118, 120, 122, 124, 126, 128, 130, 132, 134 throughout the day and night. By way of example only, the IMD 100 may measure the impedance vector every two hours throughout the day and night. The IMD 100 may determine which of the impedance vectors measured during the night (such as 10 p.m. to 6 a.m.) is the smallest of the impedance vectors. The smallest impedance vector obtained during the night may be obtained when the patient is likely to be supine and corresponding to a period of time when intracardiac and intrathoracic fluid volumes have reached a maximal state during the night. The IMD 100 may then calculate the supine chronic admittance A_{S} from the impedance vector using Equation 1 above. In another embodiment, the supine chronic admittance A_{S} may be calculated based on two or more impedance vectors and/or is based on an impedance vector that is not the smallest impedance vector measured over a time window. By way of example only, the supine chronic admittance A_{S} may be one or more of a mean, median, deviation, and the like, of several impedance vectors obtained when the patient is likely to be supine.

At 404, an upright chronic admittance (A_{U}) is measured between the predetermined combination of electrodes 104, 116, 118, 120, 122, 124, 126, 128, 130, 132, 134 (shown in FIG. 1) when the patient is in the position of a second posture that differs from the first posture. The upright chronic admittance A_{U} may be obtained by measuring the impedance vector between the predetermined combination of electrodes 104, 116, 118, 120, 122, 124, 126, 128, 130, 132, 134 after the patient has moved to the second posture for a sufficiently long time period that fluids within the patient's body have reached a steady state. For example, the upright chronic admittance A_{U} may be measured after a sufficient time to allow the fluid in the various chambers of the heart 102 (shown in FIG. 1) and other thoracic chambers to reach a steady state after the patient has moved to the second posture. In one embodiment, the second posture is an upright standing position, such as when the patient is vertically standing or sitting.

The upright chronic admittance A_{U} may be obtained by measuring the impedance vector between the predetermined combination of electrodes 104, 116, 118, 120, 122, 124, 126, 128, 130, 132, 134 after the patient have moved to the second posture and generally remained in the second posture for at least four hours. Alternatively, the upright chronic admittance A_{U} may be obtained after the patient has moved to the second posture for a different time period, such as one hour, two hours, five hours, and the like.

The upright chronic admittance A_{U} may be measured as the largest impedance vector between the predetermined combination of electrodes 104, 116, 118, 120, 122, 124, 126, 128, 130, 132, 134 (shown in FIG. 1) over a time window. As described above, the IMD 100 (shown in FIG. 1) may periodically measure the impedance vector between the predetermined combination of electrodes 104, 116, 118, 120, 122, 124, 126, 128, 130, 132, 134 throughout the day and night. The IMD 100 may determine which of the impedance vectors measured during the day (such as 6 a.m. to 6 p.m.) is the largest of the impedance vectors. The impedance vector obtained during the day may be obtained when the patient is likely to be upright and corresponding to a period of time when intracardiac and intrathoracic fluid volumes have reached a minimum state during the day. The IMD 100 may then calculate the upright chronic admittance A_{U} from the impedance vector using Equation 1 above. In another embodiment, the upright chronic admittance A_{U} may be based on two or more impedance vectors and/or on one or more impedance vectors that are not the largest impedance vector measured over a time period. By way of example only, the upright chronic admittance A_{U} may be calculated as one or more of a mean, median, deviation, and the like, of several impedance vectors obtained when the patient is likely to be upright.

At 406, a supine acute admittance (a_{S}) is measured between the predetermined combination of electrodes 104, 116, 118, 120, 122, 124, 126, 128, 130, 132, 134 (shown in FIG. 1) after the patient transitions to the first posture. The supine acute admittance a_{S} may be obtained in an in-clinic setting, such as a physician's office or hospital, by measuring the impedance vector between the predetermined combination of electrodes 104, 116, 118, 120, 122, 124, 126, 128, 130, 132, 134 shortly after the patient has moved to the first posture. By way of example only, the supine acute admittance a_{S} may be measured within a sufficiently short time period after the patient transitions from an upright standing posture to a supine posture such that fluids within the various fluid compartments have not have had a chance to equilibrate and the fluid volume within the slower responding interstitial space has not reached a steady state. However, a sufficient amount of time has elapsed to acutely alter the interelectrode spacing and to permit the fast responding intravascular fluid volume to reach a new steady state. For example, the supine acute admittance a_{S} may be measured after the patient lies down and before the fluid in the various chambers of the heart 102 (shown in FIG. 1) and other thoracic chambers reaches equilibrium.

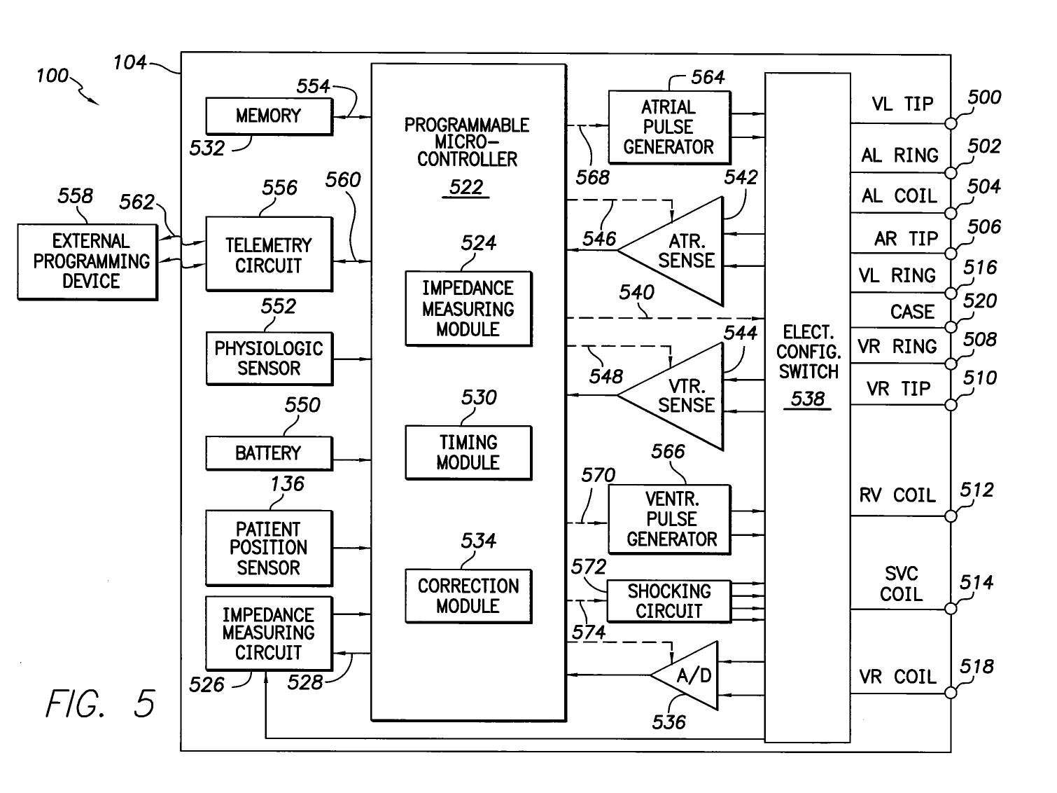

The supine acute admittance a_{S} may be measured by a physician using the IMD 100 (shown in FIG. 1). The physician may use an external device 558 (shown in FIG. 5) to direct the IMD 100 to obtain the supine acute admittance a_{S} shortly after the patient has moved to the first posture, such as within a predetermined time window after the patient has moved to the first posture. The supine acute admittance a_{S} may be based on the smallest impedance vector measured shortly after the patient has moved to the first posture which corresponds to a state when intravascular fluid volume may have reached a new maximum over a predetermined time period following the change in posture. Alternatively, the supine acute admittance a_{S} may be based on two or more impedance vectors and/or on an impedance vector that is not the smallest impedance vector measured within a time window after the patient moves to the first posture. In one embodiment, the supine acute admittance a_{S} may be measured by measuring the impedance vector between the predetermined combination of electrodes 104, 116, 118, 120, 122, 124, 126, 128, 130, 132, 134 within one minute after the patient have moved to the first posture. Alternatively, the supine acute admittance a_{S} may be obtained within a different time period after the patient has moved to the first posture, such as within 40 seconds, 30 minutes, one hour, two hours, and the like. In another embodiment, the supine acute admittance a_{S} may be calculated as one or more of a mean, median, deviation, and the like, of several impedance vectors obtained when the patient is in a supine position.

At 408, an upright acute admittance (a_{U}) is measured between the predetermined combination of electrodes 104, 116, 118, 120, 122, 124, 126, 128, 130, 132, 134 (shown in FIG. 1) after the patient moves to the second posture. Similar to the supine acute admittance a_{S}, the upright acute admittance a_{U} may be obtained in an in-clinic setting by measuring the impedance vector between the predetermined combination of electrodes 104, 116, 118, 120, 122, 124, 126, 128, 130, 132, 134 shortly after the patient has moved to the second posture, such as within a predetermined time period of moving to the second posture. By way of example only, the upright acute admittance a_{U} may be measured within a sufficiently short time period after the patient moves from a supine posture to an upright posture such that fluids within the various fluid compartments have not have had a chance to equilibrate and the fluid volume within the slower responding interstitial space has not reached a steady state. However, a sufficient amount of time has elapsed to acutely alter the interelectrode spacing and to permit the fast responding intravascular fluid volume to reach a new steady state. For example, the upright acute admittance a_{U} may be measured after the patient stands up from a supine position and before the fluid in the various chambers of the heart 102 (shown in FIG. 1) and other thoracic chambers equilibrate.

The upright acute admittance a_{U} may be measured by a physician using the IMD 100 (shown in FIG. 1). The physician may use the external device 558 (shown in FIG. 5) to direct the IMD 100 to obtain the upright acute admittance a_{U} shortly after the patient has moved to the second posture. In one embodiment, the upright acute admittance a_{U} may be based on the largest impedance vector between the predetermined combination of electrodes 104, 116, 118, 120, 122, 124, 126, 128, 130, 132, 134 within one minute after the patient have moved to the second posture which corresponds to a state when intravascular fluid volume may have reached a new minimum during a predetermined time period following a change in posture. Alternatively, the upright acute admittance a_{U} may be obtained within a different time period after the patient has moved to the second posture, such as within 40 seconds, 30 minutes, one hour, two hours, and the like. In another embodiment, the upright acute admittance a_{U} may be based on two or more impedance vectors and/or an impedance vector that is not the largest impedance vector within the time window. For example, the upright acute admittance a_{U} may be calculated as one or more of a mean, median, deviation, and the like, of several impedance vectors obtained when the patient is upright.

At 410, the offset factor β is derived for the predetermined combination of electrodes 104, 116, 118, 120, 122, 124, 126, 128, 130, 132, 134 (shown in FIG. 1) and for the movement of the patient from the first posture to the second posture. The offset factor β is based on the supine chronic and acute admittances (A_{S} and a_{S}) and the upright chronic and acute admittances (A_{U} and a_{U}). For example, the offset factor β may be based on chronic and acute changes in impedance vectors that are measured when the patient moves between postures.

In a patient where no offset factor β is needed to correct impedance vectors obtained from the predetermined combination of electrodes 104, 116, 118, 120, 122, 124, 126, 128, 130, 132, 134, the following relationship may apply between the chronic and acute admittances A_{S}, A_{U}, a_{S}, a_{U}:

ΔA=C×Δa (Eqn. 2)

where AA represents a difference between the chronic admittances (A_{S}, A_{U}), C represents an adjustment factor, and Δa represents a difference between the acute admittances (a_{S}, a_{U}). In one embodiment, the relationship shown in Equation 2 may be represented as follows:

A_{S}−A_{U}=C×(a_{S}−a_{U}) (Eqn. 3)

In one embodiment, the adjustment factor C has a value of 4 which represents the relative ratio between the fluid volume distributed in both the intravascular and interstitial fluid compartments and the fluid volume distributed in the intravascular fluid compartment alone. Alternatively, the adjustment factor C may have a different value, such as a value between 3 and 5. The adjustment factor C may be similar to the adjustment factor described in U.S. Patent Application Publication No. 2008/0262361, entitled “System and Method for Calibrating Cardiac Pressure Measurements Derived From Signals Detected by an Implantable Medical Device.”

The left side of Equation 3 represents the change between the measured chronic supine and upright admittances after a sufficient amount of time has allowed the various fluid compartments to equilibrate following the posture change, while the right side of Equation 3 represents the change between the measured acute supine and upright admittances multiplied by C after a sufficient amount of time has allowed only the intravascular fluid compartment to reach a new steady state. It is assumed here that the measured admittances are proportional to the corresponding fluid volumes within the various compartments. The factor C may be defined to represent the relative fluid volume ratio between the combined intravascular and interstitial fluid compartments and the intravascular fluid compartment alone.

Using the relationship between the admittances A_{S}, A_{U}, a_{S}, a_{U} and the impedance vectors shown above in Equation 1, Equation 3 may be expressed as follows:

$$\frac{1000}{{Z}_{S}} - \frac{1000}{{Z}_{U}} = ( \frac{1000}{{ζ}_{S}} - \frac{1000}{{ζ}_{U}} ) \times C \qquad ( Eqn . 4 )$$

where Z_{S} is the impedance vector that corresponds to the supine chronic admittance A_{S}; Z_{U} is the impedance vector that corresponds to the upright chronic admittance A_{U}; ζ_{S} is the impedance vector that corresponds to the supine acute admittance a_{S}; and ζ_{U} is the impedance vector that corresponds to the upright acute admittance a_{U}.

In a patient where the offset factor β is needed to correct impedance vectors measured by the IMD 100 (shown in FIG. 1), however, the offset factor β is included in the relationship between the impedance vectors that are associated with the chronic and acute admittances A_{S}, A_{U}, a_{S}, a_{U} set forth above in Equation 4. For example, the offset factor β adjusts impedance vectors that are affected by the patient moving to the second posture, such as an upright position. In one embodiment, the relationship shown above in Equation 4 is changed to reduce the impedance vectors obtained when the patient is in the second posture, or an upright position, by the offset factor β:

$$\frac{1}{{Z}_{S}} - \frac{1}{( {Z}_{U} - β )} = \frac{C}{{ζ}_{S}} - \frac{C}{( {ζ}_{U} - β )} \qquad ( Eqn . 5 )$$

A quadratic equation solution is used to solve for the potential values of the offset factor β appearing in Equation 5. In one embodiment, the potential values of the offset factor β may be represented by the following relationship:

$$β = \frac{- b \pm \sqrt{{b}^{2} - 4 a c}}{2 a} \qquad ( Eqn . 6 )$$

where a, b, and c are defined by the following relationships:

$$\begin{matrix}a = ( \frac{4 {Z}_{S}}{{ζ}_{S}} - 1 ) & ( Eqn . 7 ) \\ b = Δ Z + {ζ}_{U} - \frac{4 \cdot {Z}_{S}}{{ζ}_{S}} ( Δ ζ + {Z}_{U} ) & ( Eqn . 8 ) \\ c = \frac{4 \cdot {Z}_{S}}{{ζ}_{S}} ( Δ ζ * {Z}_{U} ) - Δ Z * {ζ}_{U} & ( Eqn . 9 )\end{matrix}$$

In Equations 7 through 9, ΔZ represents a difference between Z_{U} and Z_{S} and Δζ represents a difference between ζ_{U} and ζ_{S}. The values for the offset factor β may be expressed in terms of ohms. Two values may be determined from the quadratic equation solution shown above in Equations 6 through 9.

At 412, one of the two values for the offset factor β is used to adjust admittance measurements or impedance vectors obtained between the predetermined combination of electrodes 104, 116, 118, 120, 122, 124, 126, 128, 130, 132, 134 (shown in FIG. 1) when the patient moves to the second posture during a change in position of the patient or during patient activity. In one embodiment, the lower of the two values that are calculated from Equation 5 is used for the offset factor β. Alternatively, the larger of the two values may be used. For example, if the offset factor β is derived from impedance vectors 138 (shown in FIG. 1) between the LV ring electrode 134 (shown in FIG. 1) and the housing 104 when the patient moves from a first supine posterior posture to a second upright posture, then the offset factor β may be added to future impedance vectors 138 measured between the LV ring electrode 134 and the housing 104 when the patient moves from a supine posture to an upright standing posture. As described above, different offset factors β may be derived for different electrode 104, 116, 118, 120, 122, 124, 126, 128, 130, 132, 134 combinations and/or different changes in posture.

Table 1 shown below includes several offset factors β that are derived to adjust impedance vectors obtained between several different combinations of electrodes 104, 116, 118, 120, 122, 124, 126, 128, 130, 132, 134 (shown in FIG. 1) when the patient moves from a supine posture to an upright standing posture. Different tables of the offset factor β may be derived for different changes in posture by the patient. For example, a table may include the offset factors β that are applied to impedance vectors when the patient moves from a supine posture to an upright standing posture.

| Electrode | | | Offset Factor β |
|---|---|---|---|
| Combination | Electrode #1 | Electrode #2 | (ohms) |
| A | LV ring electrode 134 | Housing 104 | β₁ |
| B | RV coil electrode 130 | Housing 104 | β₂ |
| C | SVC coil electrode | Housing 104 | β₃ |
| | 132 | | |
| D | LV tip electrode 126 | RV tip electrode | β₄ |
| | | 120 | |

By way of example only, Table 1 shows that the offset factor β₁ may be subtracted from the impedance vectors obtained using the “A” combination of electrodes 104, 134 (shown in FIG. 1) when the patient transitions from the supine posture to the upright standing posture. The offset factor β₂ is added to impedance vectors obtained using the “B” combination of electrodes 130, 104, the offset factor β₃ is added to impedance vectors measured using the “C” combination of electrodes 104, 132 (shown in FIG. 1), and the offset factor β₄ is added to impedance vectors measured using the “D” combination of electrodes 120, 126 (shown in FIG. 1) when the patient transitions from the supine posture to the upright standing posture or when the patient's activity results in changing postures from the supine posture to the upright standing posture.

FIG. 5 illustrates a block diagram of exemplary internal components of the IMD 100 in accordance with one embodiment. The IMD 100 includes the housing 104 that includes an LV tip input terminal (V_{L} TIP) 500, an LA ring input terminal (A_{L} RING) 502, an LA coil input terminal (A_{L} COIL) 504, an RA tip input terminal (A_{R} TIP) 506, a right ventricular ring input terminal (V_{R} RING) 508, an RV tip input terminal (V_{R} TIP) 510, an RV coil input terminal 512, an SVC coil input terminal 514, an LV ring input terminal (V_{L} RING) 516, and an RV coil input terminal (V_{R} COIL) 518. A case input terminal 520 may be coupled with the housing 104. The input terminals 500, 502, 504, 506, 508, 510, 512, 514, 516, 518 may be electrically coupled with the electrodes 116, 118, 120, 122, 124, 126, 128, 130, 132, 134 (shown in FIG. 1).

The IMD 100 includes a programmable microcontroller 522, which controls the operation of the IMD 100. The microcontroller 522 (also referred to herein as a processor, processor module, or unit) typically includes a microprocessor, or equivalent control circuitry, and may be specifically designed for controlling the delivery of stimulation therapy and may further include RAM or ROM memory, logic and timing circuitry, state machine circuitry, and I/O circuitry. The microcontroller 522 may include one or more modules and processors configured to perform one or more of the operations described above in connection with the method 400 (shown in FIG. 4).

An impedance measurement module 524 obtains impedance vectors between predetermined combinations of the electrodes 104, 116, 118, 120, 122, 124, 126, 128, 130, 132, 134 (shown in FIG. 1). The impedance measurement module 524 communicates with an impedance measurement circuit 526 by way of a control signal 528 to control which of the electrodes 104, 116, 118, 120, 122, 124, 126, 128, 130, 132, 134 are used to obtain an impedance vector. The impedance measuring circuit 526 may be electrically coupled to a switch 538 so that an impedance vector between any desired combination of the electrodes 104, 116, 118, 120, 122, 124, 126, 128, 130, 132, 134 may be obtained.

A timing module 530 associates sampling times with impedance vectors. A sampling time is a time of the day, such as 2 a.m., that is associated with a time at which the impedance measurement module 524 obtains an impedance vector from a predetermined combination of the electrodes 104, 116, 118, 120, 122, 124, 126, 128, 130, 132, 134 (shown in FIG. 1). The timing module 530 may place or associate the impedance vectors with time stamps that indicate when each impedance vector was obtained. The time stamps and impedance vectors may be stored in and accessible from a tangible and non-transitory computer readable storage medium, such as a memory 532.

A correction module 534 adjusts the impedance vectors obtained by the impedance measuring module 524. As described above, the correction module 534 may adjust the impedance vectors by the offset factor β when the patient changes postures. In one embodiment, the correction module 534 obtains the value of the offset factor β to be applied to impedance vectors measured between a predetermined combination of the electrodes 104, 116, 118, 120, 122, 124, 126, 128, 130, 132, 134 (shown in FIG. 1) from the memory 532. Alternatively, the correction module 534 may derive the value or values of the offset factor β based on previously acquired impedance vectors, as described above. The correction module 534 communicates with the patient position sensor 136 in order to determine the postures of the patient. For example, the correction module 534 may communicate with the sensor 136 to determine the previous posture of a patient and the current posture of the patient in order to determine which offset factor β to apply to the impedance vectors.

The microprocessor 522 receives signals from the electrodes 116, 118, 120, 122, 124, 126, 128, 130, 132, 134 (shown in FIG. 1) via an analog-to-digital (A/D) data acquisition system 536. Cardiac signals obtained by the electrodes 104, 116, 118, 120, 122, 124, 126, 128, 130, 132, 134 and communicated to the data acquisition system 546. The cardiac signals are communicated through the input terminals 500, 502, 504, 506, 508, 510, 512, 514, 516, 518, 520 to an electronically configured switch bank, or switch, 538 before being received by the data acquisition system 536. Impedance vectors are obtained by the electrodes 104, 116, 118, 120, 122, 124, 126, 128, 130, 132, 134 and communicated to the impedance measuring circuit 526 via the input terminals 500, 502, 504, 506, 508, 510, 512, 514, 516, 518, 520 and switch 538.

The switch 538 includes a plurality of switches for connecting the desired electrodes 104, 116, 118, 120, 122, 124, 126, 128, 130, 132, 134 (shown in FIG. 1) and input terminals 500, 502, 504, 506, 508, 510, 512, 514, 516, 518, 520 to the appropriate I/O circuits. The switch 538 closes and opens switches to provide electrically conductive paths between the circuitry of the IMD 100 and the input terminals 500, 502, 504, 506, 508, 510, 512, 514, 516, 518, 520 in response to a control signal 540. An atrial sensing circuit 542 and a ventricular sensing circuit 544 may be selectively coupled to the leads 110, 112, 114 (shown in FIG. 1) of the IMD 100 through the switch 538 for detecting the presence of cardiac activity in the chambers of the heart 102 (shown in FIG. 1). The sensing circuits 542, 544 may sense the cardiac signals that are analyzed by the microcontroller 522. Control signals 546, 548 from the microcontroller 522 direct output of the sensing circuits 542, 544 that are connected to the microcontroller 522.

The IMD 100 additionally includes a battery 550 that provides operating power to the circuits shown within the housing 104, including the microcontroller 522. The IMD 100 may include a physiologic sensor 552 that may be used to adjust pacing stimulation rate according to the exercise state of the patient.

The memory 532 may be embodied in a tangible computer-readable storage medium such as a ROM, RAM, flash memory, or other type of memory. The microcontroller 522 is coupled to the memory 532 by a data/address bus 554. The memory 532 may store programmable operating parameters used by the microcontroller 522, as required, in order to customize the operation of IMD 100 to suit the needs of a particular patient. For example, the memory 532 may store values of the offset factor β for impedance vectors obtained using different combinations of the electrodes 104, 116, 118, 120, 122, 124, 126, 128, 130, 132, 134 (shown in FIG. 1) and/or for the patient switching between different postures. The memory 532 may store impedance vectors and/or admittances measured by the IMD 100 along with the time stamps associated with the vectors and/or impedances. The operating parameters of the IMD 100 and offset factors β may be non-invasively programmed into the memory 532 through a telemetry circuit 556 in communication with an external device 558, such as a trans-telephonic transceiver or a diagnostic system analyzer. The telemetry circuit 556 is activated by the microcontroller 522 by a control signal 560. The telemetry circuit 556 allows data and status information relating to the operation of IMD 100 to be sent to the external device 558 through an established communication link 562.

An atrial pulse generator 564 and a ventricular pulse generator 566 generate pacing stimulation pulses for delivery by the IMD 100 via the switch bank 538. The pulse generators 564, 566 are controlled by the microcontroller 522 via appropriate control signals 568, 570 respectively, to trigger or inhibit the stimulation pulses. To provide the function of an implantable cardioverter/defibrillator (ICD), the microcontroller 522 may control a shocking circuit 572 by way of a control signal 574. The shocking pulses are applied to the patient's heart 102 (shown in FIG. 1) through at least two shocking electrodes, such as the LA coil electrode 124 (shown in FIG. 1), the RV coil electrode 130 (shown in FIG. 1), and/or the SVC coil electrode 132 (shown in FIG. 1).

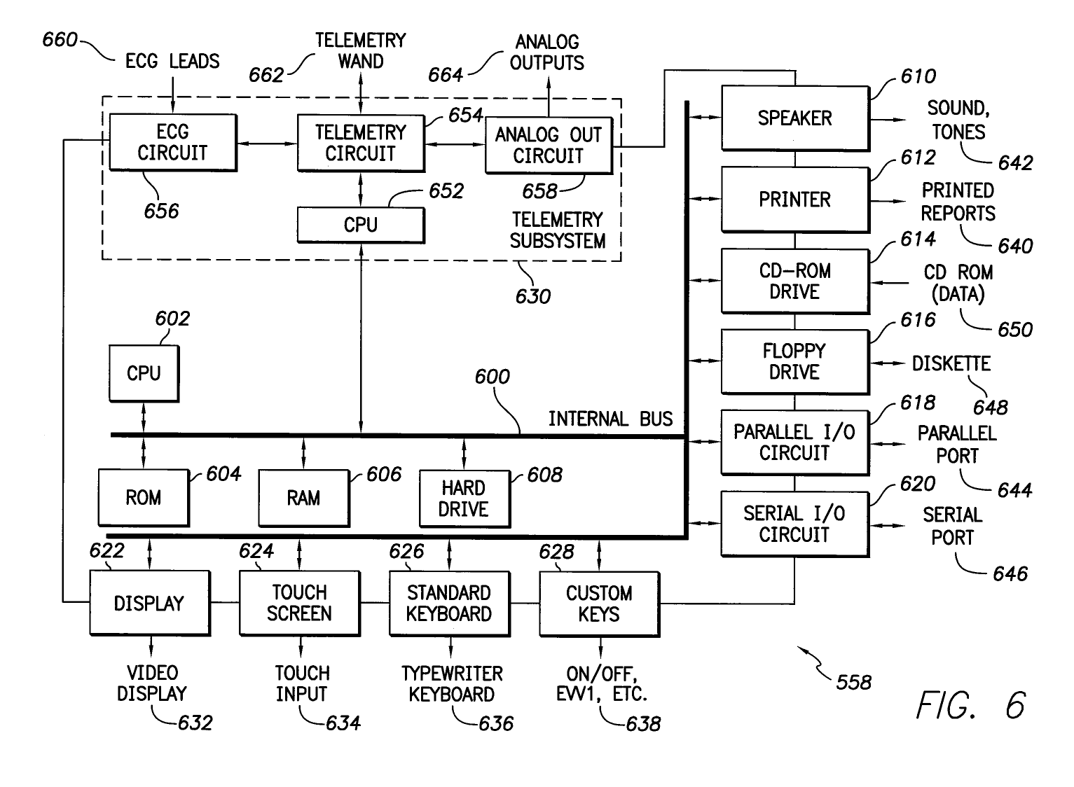

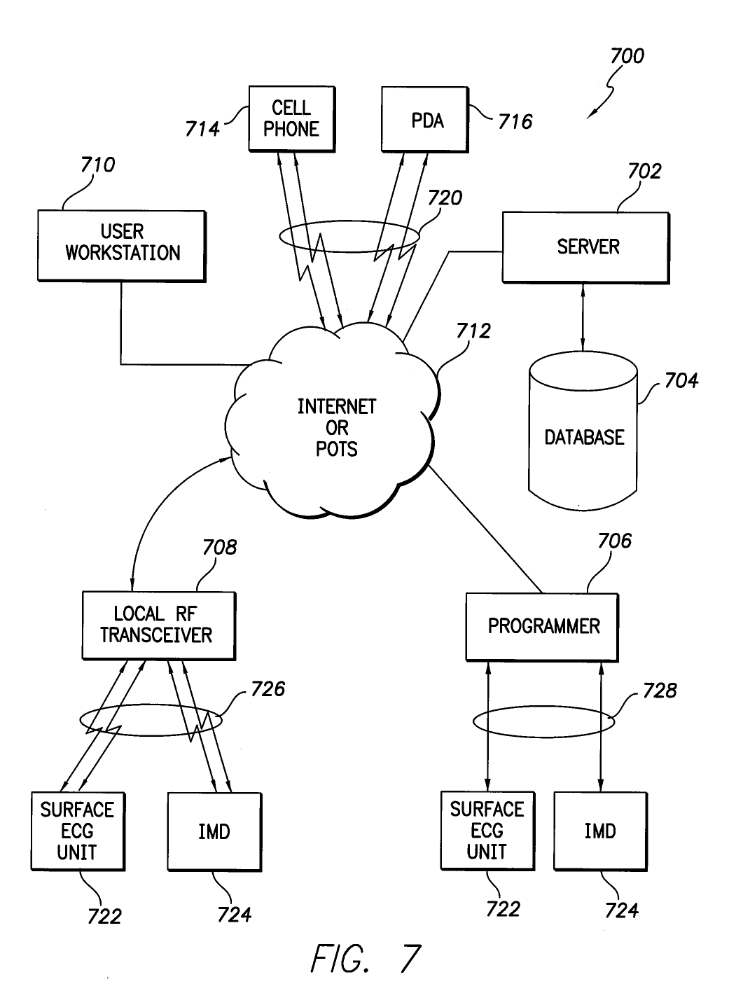

FIG. 6 illustrates a functional block diagram of the external programming device 558, such as a programmer, that is operated by a physician, a health care worker, or a patient to interface with IMD 100 (shown in FIG. 1). The external device 558 may be utilized in a hospital setting, a physician's office, or even the patient's home to communicate with the IMD 100 to change a variety of operational parameters regarding the therapy provided by the IMD 100 as well as to select among physiological parameters to be monitored and recorded by the IMD 100. For example, the external device 558 may be used to program or update offset factors β stored in the memory 532 (shown in FIG. 5) of the IMD 100 and that are used in conjunction with impedance vectors obtained by different combinations of the electrodes 104, 116, 118, 120, 122, 124, 126, 128, 130, 132, 134 (shown in FIG. 1). The external device 532 may receive impedance vectors obtained by the IMD 100 in order to calculate offset factors (3.

The external device 558 includes an internal bus 600 that connects/interfaces with a Central Processing Unit (CPU) 602, ROM 604, RAM 606, a hard drive 608, a speaker 610, a printer 612, a CD-ROM or DVD drive 614, a floppy or disk drive 616, a parallel I/O circuit 618, a serial I/O circuit 620, a display 622, a touch screen 624, a standard keyboard connection 626, custom keys 628, and a telemetry subsystem 630. The internal bus 600 is an address/data bus that transfers information (for example, either memory data or a memory address from which data will be either stored or retrieved) between the various components described. The hard drive 608 may store operational programs as well as data, such as offset factors β and the like.

The CPU 602 typically includes a microprocessor, a micro-controller, or equivalent control circuitry, designed specifically to control interfacing with the external device 558 and with the IMD 100 (shown in FIG. 1). The CPU 602 may further include RAM or ROM memory, logic and timing circuitry, state machine circuitry, and I/O circuitry to interface with the IMD 100. Typically, the microcontroller 522 (shown in FIG. 5) includes the ability to process or monitor input signals (for example, data) as controlled by program code stored in memory (for example, ROM 604).

The display 622 (for example, may be connected to a video display 632) and the touch screen 624 display text, alphanumeric information, data and graphic information via a series of menu choices to be selected by the user relating to the IMD 100 (shown in FIG. 1), such as for example, status information, operating parameters, therapy parameters, patient status, access settings, software programming version, offset factors β, impedance vectors, admittances, thresholds, and the like. The touch screen 624 accepts a user's touch input 634 when selections are made. The keyboard 626 (for example, a typewriter keyboard 636) allows the user to enter data to the displayed fields, operational parameters, therapy parameters, as well as interface with the telemetry subsystem 630. Furthermore, custom keys 628 turn on/off 638 (for example, EVVI) the external device 558. The printer 612 prints hard-copies of reports 640 for a physician/healthcare worker to review or to be placed in a patient file, and speaker 610 provides an audible warning (for example, sounds and tones 642) to the user in the event a patient has any abnormal physiological condition occur while the external device 558 is being used. The parallel I/O circuit 618 interfaces with a parallel port 644. The serial I/O circuit 620 interfaces with a serial port 646. The drive 616 accepts disks or diskettes 648. The drive 614 accepts CD and/or DVD ROMs 650.

The telemetry subsystem 630 includes a central processing unit (CPU) 652 in electrical communication with a telemetry circuit 654, which communicates with both an ECG circuit 656 and an analog out circuit 658. The ECG circuit 656 is connected to ECG leads 660. The telemetry circuit 654 is connected to a telemetry wand 662. The analog out circuit 630 includes communication circuits, such as a transmitting antenna, modulation and demodulation stages (not shown), as well as transmitting and receiving stages (not shown) to communicate with analog outputs 664. The external device 558 may wirelessly communicate with the IMD 100 (shown in FIG. 1) and utilize protocols, such as Bluetooth, GSM, infrared wireless LANs, HIPERLAN, 3G, satellite, as well as circuit and packet data protocols, and the like. A wireless RF link utilizes a carrier signal that is selected to be safe for physiologic transmission through a human being and is below the frequencies associated with wireless radio frequency transmission. Alternatively, a hard-wired connection may be used to connect the external device 558 to the IMD 100 (for example, an electrical cable having a USB connection).

FIG. 7 illustrates a distributed processing system 700 in accordance with one embodiment. The distributed processing system 700 includes a server 702 that is connected to a database 704, a programmer 706 that may similar to the external device 558 described above and shown in FIG. 5), a local RF transceiver 708, and a user workstation 710 electrically connected to a communication system 712. The communication system 712 may be an internet, the Internet or a portion thereof, a voice over IP (VoIP) gateway, a local plain old telephone service (POTS), such as a public switched telephone network (PSTN), and the like. Alternatively, the communication system 712 may be a local area network (LAN), a campus area network (CAN), a metropolitan area network (MAN), or a wide area network (WAM). The communication system 712 serves to provide a network that facilitates the transfer/receipt of cardiac signals, processed cardiac signals, histograms, trend analysis and patient status, and the like.

The server 702 is a computer system that provides services to other computing systems (for example, clients) over a computer network. The server 702 acts to control the transmission and reception of information such as cardiac signals, offset factors β, impedance vectors, admittances, statistical analysis, trend lines, and the like. The server 702 interfaces with the communication system 712, such as the internet, Internet, or a local POTS based telephone system, to transfer information between the programmer 706, the local RF transceiver 708, the user workstation 710 (as well as other components and devices) to the database 704 for storage/retrieval of records of information. By way of example only, these other components and devices may include a cell phone 714 and/or a personal data assistant (PDA) 716. The server 702 may download, via a wireless connection 720, to the cell phone 714 or the PDA 716 the results of processed cardiac signals, offset factors β, postures, impedance vectors, admittances, or a patient's physiological state based on previously recorded cardiac information, impedance vectors, postures, and the like. The server 702 may upload raw cardiac signals (for example, unprocessed cardiac data) from a surface ECG unit 722 or an IMD 724, such as the IMD 100 (shown in FIG. 1), via the local RF transceiver 708 or the programmer 706.

Database 704 is any commercially available database that stores information in a record format in electronic memory. The database 704 stores information such as raw cardiac data, processed cardiac signals, offset factors β, impedance vectors and/or admittances with associated time stamps, postures, statistical calculations (for example, averages, modes, standard deviations), histograms, and the like. The information is downloaded into the database 704 via the server 702 or, alternatively, the information is uploaded to the server 702 from the database 704.

The programmer 706 may be similar to the external device 558 shown in FIG. 5 and described above, and may reside in a patient's home, a hospital, or a physician's office. The programmer 706 interfaces with the surface ECG unit 722 and the IMD 724. The programmer 706 may wirelessly communicate with the IMD 724 and utilize protocols, such as Bluetooth, GSM, infrared wireless LANs, HIPERLAN, 3G, satellite, as well as circuit and packet data protocols, and the like. Alternatively, a hard-wired connection may be used to connect the programmer 706 to IMD 724 (for example, an electrical cable having a USB connection). The programmer 706 is able to acquire cardiac signals from the surface of a person (for example, ECGs), or the programmer 706 is able to acquire intra-cardiac electrogram (for example, IEGM) signals from the IMD 724. The programmer 706 interfaces with the communication system 712, either via the internet, Internet, and/or via POTS, to upload the data acquired from the surface ECG unit 722 or the IMD 724 to the server 702.

The local RF transceiver 708 interfaces with the communication system 712 to upload data acquired from the surface ECG unit 722 or the IMD 724 to the server 702. In one embodiment, the surface ECG unit 722 and the IMD 724 have a bi-directional connection with the local RF transceiver 708 and/or programmer 706 via a wireless connection 726, 728. The local RF transceiver 708 is able to acquire cardiac signals from the surface of a person (for example, ECGs), or acquire data from the IMD 724. On the other hand, the local RF transceiver 708 may download stored data from the database 704 or the IMD 724.

The user workstation 710 may interface with the communication system 712 to download data via the server 702 from the database 704. Alternatively, the user workstation 710 may download raw data from the surface ECG unit 722 or IMD 724 via either the programmer 706 or the local RF transceiver 708. Once the user workstation 710 has downloaded the data (for example, raw cardiac signals, impedance vectors and/or admittances with associated time stamps, offset factors β, postures, and the like), the user workstation 710 may process the data. For example, the user workstation 710 may be used to calculate various offset factors β for different combinations of electrodes and/or posture changes, as described above. Once the user workstation 710 has finished performing its calculations, the user workstation 710 may either download the results to the IMD 724 via the local RF transceiver 708 and/or programmer 706, the cell phone 714, the PDA 716, or to the server 702 to be stored on the database 704.

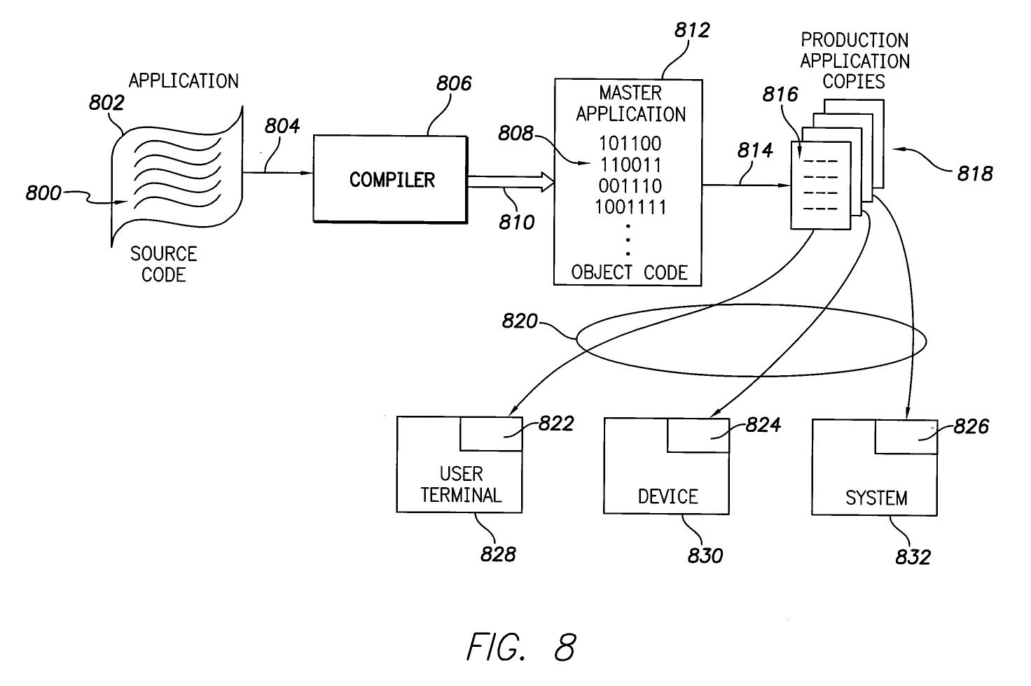

FIG. 8 illustrates a block diagram of exemplary manners in which embodiments of the present invention may be stored, distributed and installed on a tangible and non-transitory computer-readable medium. In FIG. 8, the “application” represents one or more of the methods and process operations discussed above. For example, the application may represent the processes carried out in connection with FIGS. 1 through 7 as discussed above.

As shown in FIG. 8, the application is initially generated and stored as source code 800 on a tangible and non-transitory source computer-readable medium 802. The source code 800 is then conveyed over path 804 and processed by a compiler 806 to produce object code 808. The object code 808 is conveyed over path 810 and saved as one or more application masters on a tangible and non-transitory master computer-readable medium 812. The object code 808 may then be copied numerous times, as denoted by path 814, to produce production application copies 816 that are saved on separate tangible and non-transitory production computer-readable media 818. The production computer-readable media 818 are then conveyed, as denoted by path 820, to various systems, devices, terminals and the like. In the example of FIG. 8, a user terminal 822, a device 824, and a system 826 are shown as examples of hardware components, on which the production computer-readable media 818 are installed as applications (as denoted by 828, 830, 832). For example, the production computer-readable media 818 may be installed on one or more of the IMD 100 (shown in FIG. 1), the user workstation 710 (shown in FIG. 7), the server 702 (shown in FIG. 7), the database 704 (shown in FIG. 7), the cell phone 714 (shown in FIG. 7), the PDA 716 (shown in FIG. 7), the programmer 706 (shown in FIG. 7), and the like.

The source code 800 may be written as scripts, or in any high-level or low-level language. Examples of the source, master, and production computer-readable medium 802, 812, and 818 include, but are not limited to, tangible media such as CD-ROM, DVD-ROM, RAM, ROM, flash memory, RAID drives, memory on a computer system and the like. Examples of the paths 804, 810, 814, 820 include, but are not limited to, network paths, the internet, Bluetooth, GSM, infrared wireless LANs, HIPERLAN, 3G, satellite, and the like. The paths 804, 810, 814, 820 may also represent public or private carrier services that transport one or more physical copies of the source, master, or production computer-readable media 802, 812, 816 between two geographic locations. The paths 804, 810, 814, 820 may represent threads carried out by one or more processors in parallel. For example, one computer may hold the source code 800, compiler 806, and object code 808. Multiple computers may operate in parallel to produce the production application copies 816. The paths 804, 810, 814, 820 may be intra-state, inter-state, intra-country, inter-country, intra-continental, inter-continental and the like.

The operations noted in FIG. 8 may be performed in a widely distributed manner world-wide with only a portion thereof being performed in the United States. For example, the application source code 800 may be written in the United States and saved on a source computer-readable medium 802 in the United States, but transported to another country (corresponding to path 804) before compiling, copying and installation. Alternatively, the application source code 800 may be written in or outside of the United States, compiled at a compiler 806 located in the United States and saved on a master computer-readable medium 812 in the United States, but the object code 808 transported to another country (corresponding to path 814) before copying and installation. Alternatively, the application source code 800 and object code 808 may be produced in or outside of the United States, but production application copies 816 produced in or conveyed to the United States (for example, as part of a staging operation) before the production application copies 816 are installed on user terminals 822, devices 824, and/or systems 826 located in or outside the United States as applications 828, 830, 832.

As used throughout the specification and claims, the phrases “computer-readable medium” and “instructions configured to” shall refer to any one or all of (i) the source computer-readable medium 802 and source code 800, (ii) the master computer-readable medium and object code 808, (iii) the production computer-readable medium 818 and production application copies 816 and/or (iv) the applications 828, 830, 832 saved in memory in the terminal 822, device 824, and system 826.

In accordance with certain embodiments, methods, systems, and devices are provided that are able to adjust impedance vectors and/or admittances based on changes in a patient's posture. The adjustments may be used to modify the impedance vectors and/or admittances in order to compensate for posture dependent changes in the interelectrode spacing and geometry so that physiological parameters such as LAP may be estimated more accurately.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions and types of materials described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. An implantable medical device comprising:
electrodes configured to be positioned within at least one of a heart and chest wall of a patient;
an impedance measurement module to measure an impedance value between a predetermined combination of the electrodes;
a patient position sensor to determine at least one of a posture and an activity level of the patient; and
a correction module to adjust the impedance value based on the at least one of the posture and the activity level of the patient.

2. The implantable medical device of claim 1, wherein the correction module adjusts the impedance value by applying an offset factor to the impedance value, the offset factor having a value that varies based on the at least one of the posture and the activity level of the patient.

3. The implantable medical device of claim 1, wherein the correction module adjusts the impedance values by applying an offset factor to the impedance value, the offset factor based on a comparison between acute and chronic changes in previously obtained impedance values following a change in the posture of the patient.

4. The implantable medical device of claim 1, wherein the correction module adjusts the impedance values by applying an offset factor to the impedance value, the offset factor based on chronic changes in previously obtained impedance values following a change in the posture of the patient.

5. The implantable medical device of claim 4, wherein the chronic changes in the previously obtained impedance values include a difference between the previously obtained impedance values that were measured at least one hour after the change in the posture of the patient.

6. The implantable medical device of claim 1, wherein the correction module adjusts the impedance values by an offset factor, the offset factor based on acute changes in previously obtained impedance values following a change in the posture of the patient.

7. The implantable medical device of claim 6, wherein the acute changes in previously obtained impedance values include a difference between the previously obtained impedance values that were measured within one minute after the change in the posture of the patient.

8. The implantable medical device of claim 1, wherein the posture is a current posture and the correction module continues to adjust impedance values measured by the impedance measurement module between the predetermined combination of electrodes by applying an offset factor to the impedance measurements for a predetermined time period after the patient changes from a previous posture to the current posture.

9. The implantable medical device of claim 1, wherein the correction module adjusts the impedance value by selecting an offset factor from a plurality of offset factors and applying the offset factor to the impedance value, the offset factor selected from the plurality of offset factors based on the predetermined combination of electrodes used to measure the impedance value.

10. The implantable medical device of claim 1, wherein the correction module adjusts the impedance value by selecting an offset factor from a plurality of offset factors and applying the offset factor to the impedance value, the offset factor selected from the plurality of offset factors based on the at least one of the posture and the activity level of the patient.

11. The implantable medical device of claim 1, wherein the correction module uses the at least one of the posture and the activity level of the patient to adjust a left atrial pressure estimate of the patient.

12. A method for adjusting an impedance value obtained by a medical device, the method comprising:
measuring the impedance value using a predetermined combination of electrodes that are positioned in at least one of a heart and a chest wall of a patient;
determining at least one of a posture and an activity level of the patient when the impedance value is measured; and
adjusting the impedance value based on the at least one of the posture and the activity level of the patient.

13. The method of claim 12, wherein the adjusting operation comprises applying an offset factor to the impedance value, the offset factor having a value that varies based on the at least one of the posture and the activity level of the patient.

14. The method of claim 12, wherein the adjusting operation comprises applying an offset factor to the impedance value, the offset factor based on a comparison between acute and chronic changes in previous obtained impedance values following a change in the posture of the patient.

15. The method of claim 12, wherein the adjusting operation comprises applying an offset factor to the impedance value, the offset factor based on chronic changes in previously obtained impedance values following a change in the posture of the patient.

16. The method of claim 12, wherein the adjusting operation comprises applying an offset factor to the impedance value, the offset factor based on acute changes in previously obtained impedance values following a change in the posture of the patient.

17. The method of claim 12, wherein the posture is a current posture and the adjusting operation continues to adjust impedance values measured between the predetermined combination of electrodes by applying an offset factor to the impedance measurements for a predetermined time period after the patient changes from a previous posture to the current posture.

18. The method of claim 12, wherein the adjusting operation comprises selecting an offset factor from a plurality of offset factors and applying the offset factor to the impedance value, the offset factor selected from the plurality of offset factors based on the predetermined combination of electrodes used to measure the impedance value.

19. The method of claim 12, wherein the adjusting operation comprises selecting an offset factor from a plurality of offset factors and applying the offset factor to the impedance value, the offset factor selected from the plurality of offset factors based on the at least one of the posture and the activity level of the patient.

20. A system comprising:
means for measuring an impedance value using a predetermined combination of electrodes that are positioned in at least one of a heart and a chest wall of a patient;
means for determining at least one of a posture and an activity level of the patient; and
means for adjusting the impedance value based on the means for determining.
